(19) United States
(12) Patent Application Publication
(10) Pub. No.: US 2017/0224524 A1
(43) Pub. Date: Aug. 10, 2017

(54) Ostomy Appliance

(71) Applicant: Salts Healthcare Limited, Birmingham, West Midlands (GB)

(72) Inventors: Neil Wiltshire, Birmingham, West Midlands (GB); Peter Argent, Birmingham, West Midlands (GB)

(21) Appl. No.: 15/502,429

(22) PCT Filed: Aug. 6, 2015
(86) PCT No.: PCT/GB15/52276

§ 371 Date: Feb. 7, 2017

(30) Foreign Application Priority Data
Aug. 7, 2014 (GB) 1414018.0

(51) Int. Cl. A61F 5/445; A61F 5/441; A61F 5/44

(52) U.S. Cl. CPC A61F 5/445; A61F 5/44; A61F 5/441; A61F 5/4404; A61F 5/4405

(57) ABSTRACT

An ostomy appliance (10) including first and second walls (12, 14) connected to each other at or near their peripheries, the first wall having a stoma-receiving opening (16), and a waste collecting cavity defined between the first and second walls, wherein the second wall includes an outlet (20) which communicates with the cavity, and wherein the appliance includes a blocking member (50) moveable between a first position in which the outlet is blocked or substantially blocked and a second position in which the outlet is unblocked or substantially unblocked.

### DESCRIPTION OF INVENTION

This invention relates to an ostomy appliance. More particularly, this invention relates to an ostomy appliance including a gas flow regulation device.

An ostomy appliance is used for collecting human waste from a stoma, which is a surgically constructed tube of a user's digestive tract that protrudes through a user's skin. Different types of ostomy appliances are used depending on the type of stoma. If the stoma is from the small intestine (ileum) a drainable ostomy appliance is often preferred, because the waste collected is generally in liquid form and can easily be empted from the appliance. If the stoma is from the large intestine or colon, then a non-drainable ostomy appliance is preferred. This is because the collected waste is usually solid or semi-solid, and it is therefore not possible to easily empty the appliance.

A problem with ostomy appliances, particularly those which are not drainable and thus include a gas outlet (often in combination with a filter), is that ‘pancaking’ can occur during use. This is where side walls of the bag of the appliance stick together preventing waste from falling to the bottom of the bag. Waste thus collects in the upper region of the bag and can block gas outlets from the bag or even force the bag to become detached from the user.

According to a first aspect of the invention, we provide an ostomy appliance including:
first and second walls connected to each other at or near their peripheries, the first wall having a stoma-receiving opening; and
a waste collecting cavity defined between the first and second walls,
wherein the second wall includes an outlet which communicates with the cavity, and wherein the appliance includes
a blocking member moveable between a first position in which the outlet is blocked or substantially blocked and a second position in which the outlet is unblocked or substantially unblocked.

Further features of the invention are set out in claims 2 to 21 appended hereto.

According to a second aspect of the invention, we provide an ostomy appliance including:
first and second walls connected to each other at or near their peripheries, the first wall having a stoma-receiving opening; and
a waste collecting cavity defined between the first and second walls,
wherein the second wall includes an outlet which communicates with the cavity, and wherein the appliance includes:
a member moveable between a first and second positions, wherein when said member is in its first position the first and second walls are held spaced from each other.

Further features of the invention are set out in claims 23 to 42 appended hereto.

Embodiments of the various aspects of the invention will now be described by way of example only, with reference to the accompanying drawings, of which:

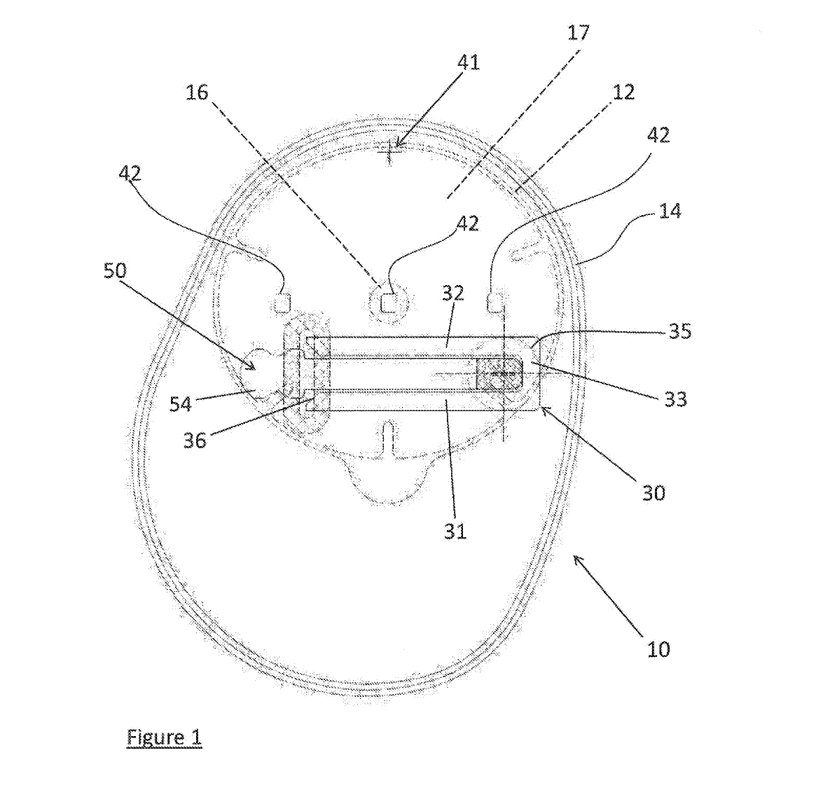

FIG. 1 is a front view of an ostomy appliance according to the present invention;

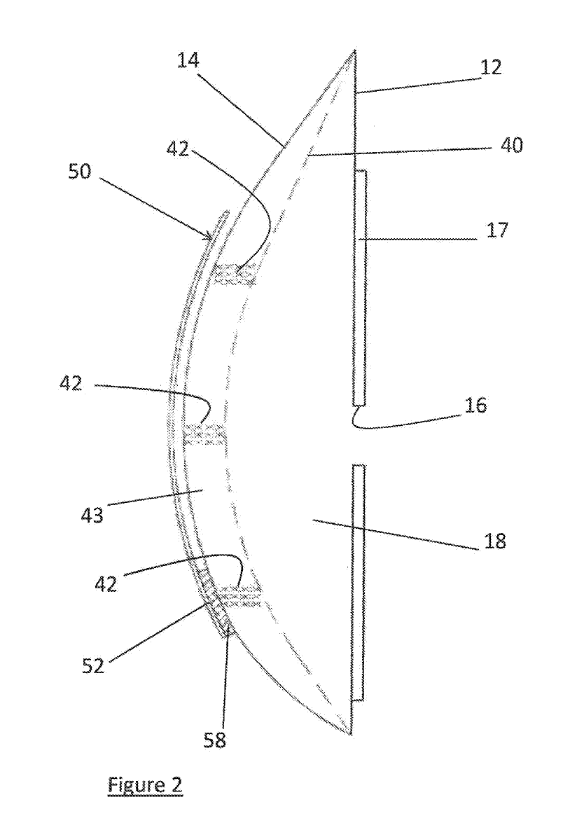

FIG. 2 is a plan view of the ostomy appliance of FIG. 1;

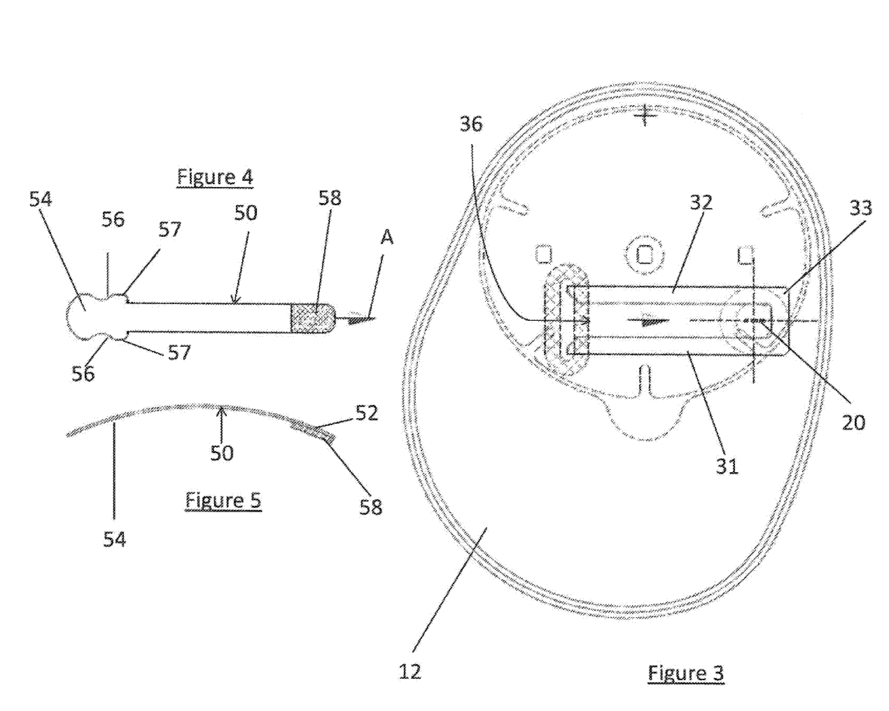

FIG. 3 is a further front view of the appliance, but with the blocking member removed;

FIG. 4 is a front view of a blocking member; and

FIG. 5 is a side view of the blocking member of FIG. 4.

Referring to the figures, these show an embodiment of an ostomy appliance 10 according to the present invention, in this example the appliance 10 is a non-drainable appliance (although it need not be, so could have a drainable outlet) which has first 12 and second 14 walls connected to each other at or near their peripheries. The walls 12, 14 are made from a suitable plastics material. The first wall 12 has a hydrocolloid member 17 connected thereto with a stoma-receiving opening 16 therein, as is well known in the art. The size of the opening 16 can be increase, typically by cutting, so that it adequately fits a user's stoma.

The appliance 10, in this embodiment includes a further, intermediate, wall 40 positioned in between the first 12 and second 14 walls. The further wall 40 is connected to the first 12 and second 14 walls at or near their peripheries. The further wall 40 and the second wall 14 are connected to each other at a further location 42 other than their peripheries. In this embodiment they are connected by three spaced heat welds 42. There could be fewer or more connections and they need not be heat welds. The purpose of the connections 42 is to hold the wall 40 relative to the second wall 14 when the latter is moved, as will become apparent later.

The wall 40 divides the space between the walls 12, 14 into first 18 and second 43 sub-cavities. The first sub-cavity 18 is the waste collecting cavity and thus communicates with the opening 18 to receive waste.

The further wall 40 includes a passage, in this case an aperture, 41, positioned in an upper region thereof, for flow of gas between the first 18 and second 43 sub-cavities.

The second wall 14 includes an outlet 20 (again, in this example an aperture in the wall) which communicates with the cavity 43, and thus indirectly with the waste cavity 18. The outlet 20 permits gases to ultimately escape from the appliance to atmosphere. In this example the outlet 20 is covered by a gas filter 35 (to remove odours from the escaping gases), but this is not essential.

The further wall 40 is not essential, and could be removed, leaving just a single cavity 18 between the walls 12, 14, in such an embodiment, however, the outlet 20 and its gas filter would have no protection from being contact by waste in the cavity 18, which could lead to blockage of the outlet 20 and ballooning/possible rupture of the appliance. However, alternatives means for preventing blockage to the filter may be provided, for example a hydrophobic covering or cover member.

As shown in the figures, the appliance 10 includes a moveable blocking member 50 for blocking or substantially blocking the outlet 20. In this example the blocking member 50 is slidably moveable, but it need not be moveable in that way. The blocking member 50 is an elongate plastics component, which is curved, i.e. non-planar, in side view (see FIG. 5). The blocking member 50 is made from a resiliency deformable material, such that it can flex. For example, it can be move to a substantially planar condition, but when any bending forces are removed, will revert to its curved position.

The blocking member 50 has a blocking end 52 and a user-graspable end 54, which are opposite each other. The blocking end 54 has connected thereto a deformable, e.g. foam, member 58, which in use faces and directly engages the outlet 20, when in the blocking condition. The member 58 may be resiliently deformable. The end 52 is shaped substantially like the body of a guitar, with opposing recesses 58 for receiving the fingers of a user. A pair of shoulder portions 57 are provided adjacent the recesses 56, the purpose of which will be seen later. Fewer or more recesses 56 may be provided.

The appliance includes a blocking member receiving part 30 which is connected to the second wall 14 by heat welding (although other connection methods could be used). The receiving part 30 could be an integral formation in the wall 14, for example. In this example the receiving part 30 is a substantially rectangular plastics sheet which is heat welded to the wall 14 along sections 31, 32 and 33 to form a U-shaped connection. The U-shaped connection forms a channel or passage 36 which extends laterally across the wall 14 and communicates with the outlet 20 and receives the blocking member 50.

The blocking member 50 is moveable between a first position (see FIG. 1) in which the outlet 20 is blocked or substantially blocked by the member 58 and a second position (see FIG. 3) in which the outlet 20 is unblocked or substantially unblocked. In FIG. 3 the blocking member 50 has been completely removed from the passage 36, so the outlet is completely unblocked. However, it could be slid partially along the passage 36 so that the flow rate of gas exiting the outlet 20 is reduced.

The curved shape of the blocking member 50 means that when the blocking member 50 is in its blocking position (FIG. 1) the first 12 and second 14 walls are held spaced from each other. As the wall 40 is connected lo the second wall 14, the wall 40 is also held spaced from the wall 12. This is illustrated in FIG. 2, where it can be seen that the second wall 14 and wall 40 adopts a curved or non-planar profile, with the walls 14, 40 curving outwardly away from the first wall 12.

The provision of the blocking member 50 in its blocking position thus holds the walls 14, 40 away from the wall 12 to prevent or at least minimise the occurrence of pancaking. However, the dual functionality of the blocking member 50 means that the user can also control the rate of flow of gas through the outlet, to maintain a desired amount of gas in the cavities 18, 43.

Whilst in the above embodiment the member 50 acts both to block the outlet and also hold the walls apart from each other, this double action is not essentially in accordance with the invention. For example, embodiments are envisaged where the blocking member may be moveable between a first position in which the outlet is blocked or substantially blocked and a second position in which the outlet is unblocked or substantially unblocked, but whilst not providing any or any reasonable amount of holding of the first and second walls are spaced apart from each other. Alternatively, a member could be provided which does not provide any blocking effect of the outlet, but which is still moveable between first and second positions, such that in its first position the first and second walls are held spaced from each other, in such an embodiment, a separate means for blocking the outlet may or may not be provided.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. An ostomy appliance including:
first and second walls connected to each other at or near their peripheries, the first wall having a stoma-receiving opening; and
a waste collecting cavity defined between the first and second walls,
wherein the second wall includes an outlet which communicates with the cavity, and wherein the appliance includes:
a blocking member moveable between a first position in which the outlet is blocked or substantially blocked and a second position in which the outlet is unblocked or substantially unblocked.

2. An appliance according to claim 1 wherein when said blocking member is in its blocking position the first and second walls are held spaced from each other.

3. An appliance according to claim 1 wherein the blocking member is slidably moveable between the first and second positions.

4. An appliance according to claim 1, including one of the following: wherein the blocking member is curved or non-planar; wherein the blocking member is elongate; wherein the blocking member is resiliently deformable.

5. (canceled)

6. (canceled)

7. An appliance according to claim 1 including one of the following: wherein, when the blocking member is in its first position, the or a portion of the second wall adopts a curved or non-planar profile; wherein, when the blocking member is in its first position, the or a portion of the second wall is curved outwardly away from the first wall.

8. (canceled)

9. An appliance according to claim 1, wherein the blocking member has a blocking end and a user-graspable end and wherein the user-graspable end is provided with one or more formations.

10. (canceled)

11. An appliance according to claim 9 wherein the or each formation is a recess.

12. An appliance according to claim 9 wherein the blocking end includes a resiliently deformable member.

13. (canceled)

14. An appliance according to claim 1 wherein the second wall includes or has connected thereto a receiving part for receiving the blocking member, wherein the receiving part extends laterally across the second wall and includes a channel or passage which communicates with the outlet.

15. (canceled)

16. (canceled)

17. An appliance according to claim 1 including a further wall positioned in between the first and second walls, and dividing the cavity into first and second sub-cavities.

18. An appliance according to claim 17 wherein the further wall is connected to the first and second walls at or near their peripheries

19. An appliance according to claim 17 wherein the further wall includes a passage for flow of gas between the first and second sub-cavities

20. An appliance according to claim 17 wherein the further wall and the second wall are connected to each other at a further location other than their peripheries.

21. (canceled)

22. An ostomy appliance including:
first and second walls connected to each other at or near their peripheries, the first wall having a stoma-receiving opening; and
a waste collecting cavity defined between the first and second walls,
wherein the second wall includes an outlet which communicates with the cavity, and wherein the appliance includes:
a member moveable between a first and second positions, wherein when said member is in its first position the first and second walls are held spaced from each other.

23. An appliance according to claim 22 wherein when in the first position the member blocks or substantially blocks the outlet and wherein when in the second position the outlet is unblocked or substantially unblocked.

24. An appliance according to claim 22 wherein the member is slidably moveable between the first and second positions.

25. An appliance according to claim 22 including one of the following: wherein the member is curved or non-planar; wherein the member is elongate; wherein the member is resiliently deformable.

26. (canceled)

27. (canceled)

28. An appliance according to claim 22 including one of the following: wherein, when the member is in its first position, the or a portion of the second wall adopts a curved or non-planar profile; wherein, when the member is in its first position, the or a portion of the second wall is curved outwardly away from the first wall.

29. (canceled)

30. An appliance according to claim 22 wherein the member has a blocking end and a user-graspable end.

31. An appliance according to claim 30 wherein the use-graspable end is provided with one or more formations, wherein the or each formation is a recess.

32. (canceled)

33. An appliance according to claim 30 wherein the end includes a resiliently deformable member.

34. (canceled)

35. An appliance according to claim 22 wherein the second wall includes or has connected thereto a receiving part for receiving the member, wherein the receiving part extends laterally across the second wall and includes a channel or passage which communicates with the outlet.

36. (canceled)

37. (canceled)

38. An appliance according to claim 22 including a further wall positioned in between the first and second walls, and dividing the cavity into first and second sub-cavities.

39. An appliance according to claim 38 wherein the further wall is connected to the first and second walls at or near their peripheries

40. An appliance according to claim 38 wherein the further wall includes a passage for flow of gas between the first and second sub-cavities

41. An appliance according to claim 38 wherein the further wall and the second wall are connected to each other at a further location other than their peripheries.

42. An appliance according to claim 38 wherein when the member is moved to its first position, the second wall and further wall are held spaced from the first wall.

43. (canceled)

44. (canceled)
